(19) United States
(12) Patent Application Publication
(10) Pub. No.: US 2024/0329028 A1
(43) Pub. Date: Oct. 3, 2024

(54) SOIL PROFILE SENSING AND SAMPLING DEVICE AND METHOD

(71) Applicant: Veris Technologies, Inc., Salina, KS (US)

(72) Inventors: Eric Lund, Salina, KS (US); Kyle Jensen, Salina, KS (US); Chase Maxton, Salina, KS (US)

(21) Appl. No.: 18/624,093

(22) Filed: Apr. 1, 2024

Related U.S. Application Data
(60) Provisional application No. 63/456,374, filed on Mar. 31, 2023.

(51) Int. Cl. G01N 33/24; G01N 1/08

(52) U.S. Cl. CPC G01N 33/246; G01N 1/08; G01N 33/243; G01N 33/245

(57) ABSTRACT

A soil profile sensing and sampling device has a soil probe and a linear actuator for applying downforce to insert the soil probe into the soil. The soil probe can be a soil sensing probe or a soil coring probe. A shuttle system is configured to allow insertion of the soil probe into the soil to a depth that exceeds a stroke length of the linear actuator. The sensing probe can be equipped with a plurality of sensors for sensing soil EC, soil optical reflectance, soil capacitance, and soil compactness. The soil data collected by the sensors can be used in a pedotransfer function to estimate soil bulk density. The soil probe can be operated in an automatic probe sampling sequence to insert the soil probe into the soil when a sampling distance interval has been met and a horizontal speed of the soil probe is approximately zero.

### RELATED APPLICATIONS

This application claims the benefit of U.S. Provisional Patent Application No. 63/456,374 filed on Mar. 31, 2023, the contents of which are incorporated herein by reference.

### BACKGROUND OF THE INVENTION

### Field of the Invention

The present invention relates generally to soil sensing and sampling devices and methods, and in particular, to soil sensing and sampling devices and methods that can be used with compact vehicles to measure inventories of soil organic carbon (SOC) and other properties in the soil.

### Description of the Related Art

Inventories of soil organic carbon (SOC) are needed to properly compensate growers who are engaging in conservation practices and marketing an anticipated carbon increase in their soils. Soil SOC varies widely with most fields and within most soil profiles. Accounting for this variability with lab-analyzed samples is not feasible. Sensors for measuring soil properties in situ in the field are needed.

Proper measurements of SOC require bulk density to be measured. SOC increases must be based on SOC (%)×bulk density (gm/cc). Measuring bulk density manually is time consuming and expensive.

Soil health initiatives also need an improved understanding of the soil profile variations. Public and private researchers need devices to help advance their understanding of soil profiles. Existing methods of determining inventories of SOC primarily still use soil sampling, although some probes with in situ sensors have been developed and used.

### SUMMARY OF THE INVENTION

An object of the present invention is to provide a soil profile sensing and sampling device that can be carried on a utility vehicle or compact tractor and used to inventory SOC and other soil properties in fields.

A further object of the present invention is to provide a soil profile sensing device that uses sensors with pedotransfer functions to eliminate a separate step of measuring bulk density.

A further object of the present invention is to provide a soil profile sensing and sampling device that has a compact configuration that allows a depth of insertion of a soil probe to exceed a stroke length of a linear actuator used to apply downforce for inserting the soil probe.

A further object of the present invention is to provide a soil profile sensing and sampling device that has an automatic probe sampling sequence that allows the soil probe to be automatically inserted and retracted at predetermined locations or sampling intervals in a field.

A further object of the present invention is to provide a soil sensing probe equipped with a variety of sensors for measuring soil properties and a pedotransfer function to estimate soil bulk density.

A further object of the present invention is to provide a compact soil probing device that automatically activates a vibration mechanism to facilitate insertion of a soil probe into the soil when encountering hard-to-penetrate soil conditions.

To accomplish these and other objects of the present invention, a soil profile sensing and sampling device and method include a soil probe and a linear actuator for applying downforce to insert the soil probe into the soil. The soil probe can be a soil sensing probe or a soil coring probe. A shuttle system is configured to allow insertion of the soil probe into the soil to a depth that exceeds a stroke length of the linear actuator. The sensing probe can be equipped with a plurality of sensors for sensing soil EC, soil optical reflectance, soil capacitance, and soil compactness. The soil data collected by the sensors can be used in a pedotransfer function to estimate soil bulk density. The soil probe can be operated in an automatic probe sampling sequence to insert the soil probe into the soil when a sampling distance interval has been met and a horizontal speed of the soil probe is approximately zero.

According to one aspect of the invention, a soil profile sensing device is provided, comprising: a soil probe equipped with a plurality of sensors for sensing a plurality of soil properties selected from the group consisting of soil EC, soil optical reflectance, soil capacitance, and soil compactness; and an automated controller that automatically inserts the soil probe into the soil upon detecting that a predetermined sampling distance interval has been met and the soil probe has stopped horizontal movement.

According to another aspect of the invention, a compact soil probing device is provided, comprising: a soil probe; a linear actuator for applying downforce to push the soil probe into soil; and a shuttle system arranged to transfer downforce from the linear actuator to the soil probe, the shuttle system having a multiple hit configuration that allows insertion of the soil probe into the soil to a depth that exceeds a stroke length of the linear actuator.

According to another aspect of the invention, a method of operating a soil probe device is provided, comprising: determining a location of the soil probe device within a field using a GPS receiver; selecting a sampling distance interval; measuring a horizontal speed of the soil probe device; initiating an automatic probe sampling sequence when the sampling distance interval has been met and the horizontal speed of the soil probe device is zero; and operating the soil probe device according to the automatic probe sampling sequence to insert a soil probe of the soil probe device into soil to sense a soil property or collect a soil core.

Numerous other objects of the present invention will be apparent to those skilled in this art from the following description wherein there is shown and described an embodiment of the present invention, simply by way of illustration of one of the modes best suited to carry out the invention. As will be realized, the invention is capable of other different embodiments, and its several details are capable of modification in various obvious aspects without departing from the invention. Accordingly, the drawings and description should be regarded as illustrative in nature and not restrictive.

### BRIEF DESCRIPTION OF THE DRAWINGS

The present invention will become more clearly appreciated as the disclosure of the present invention is made with reference to the accompanying drawings. In the drawings:

FIG. 1 is a rear perspective view of a soil probing device according to the present invention, with the soil probe in a raised transport position.

FIG. 2 is a front perspective view of the soil probing device, with the soil probe in a raised transport position.

FIG. 3 is another front perspective view of the soil probing device, with the foot compressed and the tip of the soil probe in the centering hole of the foot.

FIGS. 4(A) to 4(E) illustrate a probe sampling sequence using a double hit shuttle mechanism of the present invention, wherein: FIG. 4(A) shows the soil probe and foot in a raised transport position; FIG. 4(B) shows the foot in a compressed position against the soil surface with the soil probe in a top carrier position; FIG. 4(C) shows the soil probe in a first probe stage position; FIG. 4(D) shows the soil probe carrier moved up to lock the probe in a bottom carrier position; and FIG. 4(E) shows the final probe stage with the soil probe fully inserted into the soil.

FIGS. 5(A) to 5(E) illustrate a position sensor used in the soil probing device to monitor a vertical position of the soil probe during the probe sampling sequence.

FIG. 6 is a front elevation view of a soil profile sensing device according to the present invention, which shows an optical sensor for measuring soil organic matter.

FIG. 7 is a side elevation view of the soil profile sensing device shown in FIG. 6, which shows an optical sensor, a soil moisture sensor, and a di-pole soil EC sensor.

FIG. 8 is a rear elevation view of the soil profile sensing device shown in FIG. 6, which shows a soil moisture sensor and a downforce sensor.

FIG. 9 is a rear elevation view of the soil probing device of the present invention with a core sampler assembly for collecting soil core samples.

FIG. 10 is an exploded view of the core sampler assembly shown in FIG. 9.

FIG. 11 is a flowchart showing a probe sampling sequence according to the present invention.

FIG. 12 is a flowchart showing an erroneous log detection feature provided in the controller of the present invention.

### DETAILED DESCRIPTION OF THE INVENTION

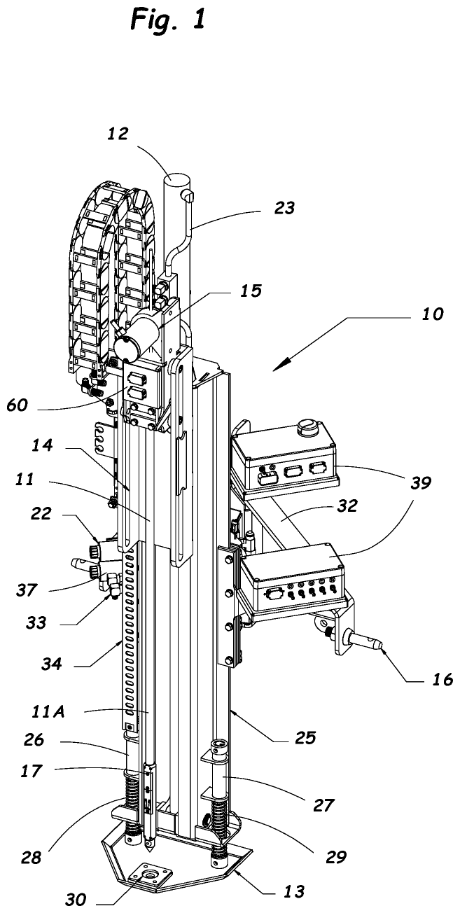

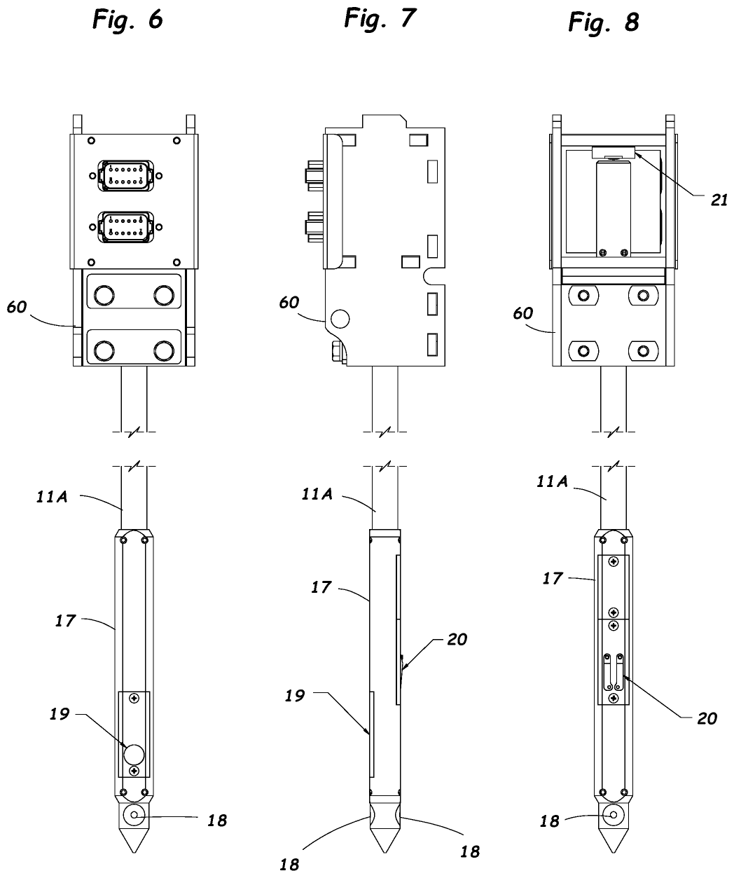

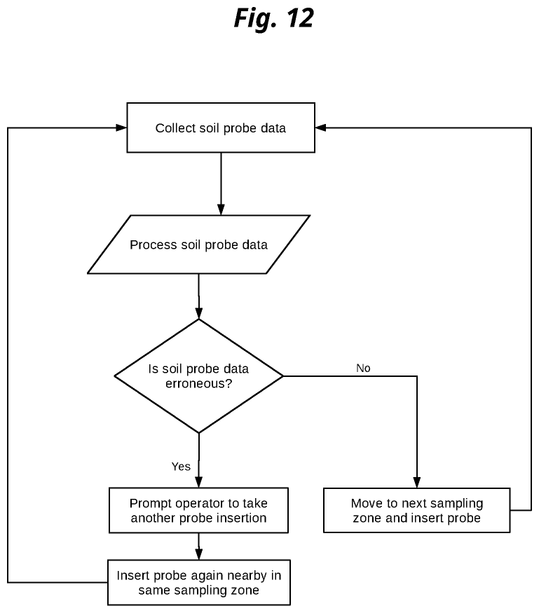

A soil profile sensing and sampling device 10 and method according to the present invention will be described in detail with reference to FIGS. 1 to 12 of the accompanying drawings.

The soil profile sensing and sampling device 10 includes a soil probe 11, a linear actuator 12 for applying downforce to push the soil probe 11 into soil, a soil engaging foot 13, a shuttle system 14 arranged to transfer downforce from the linear actuator 12 to the soil probe 11, and a vibration mechanism 15. The soil profile sensing device 10 has a lightweight, compact configuration and can be mounted to a utility vehicle or a compact tractor using, for example, a universal three-point hitch 16. The soil profile sensing device 10 can be manually operated or automated as explained below.

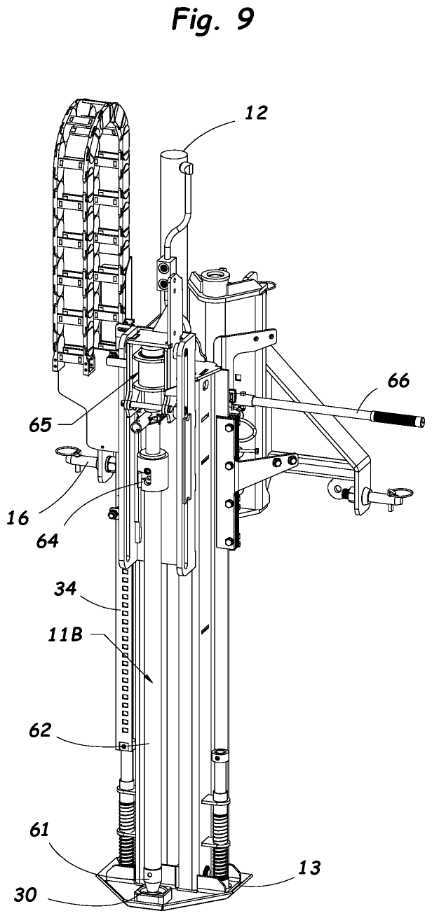

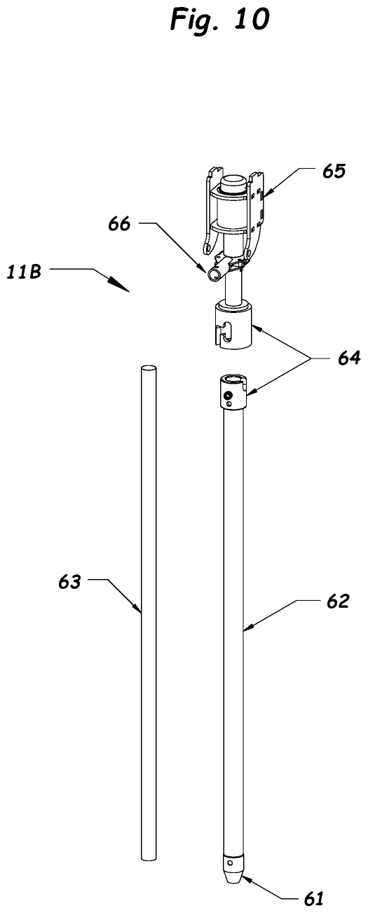

The soil probe 11 can be a soil sensor probe 11A, as illustrated in FIGS. 1 to 3 and 6 to 8, with sensors for collecting soil data as the probe is inserted to a sensing depth (e.g., approximately one meter). The soil probe 11 can also be a soil core sampling probe 11B, as illustrated in FIGS. 9 and 10, for collecting soil core samples to a similar depth. The soil sensor probe 11A and the soil core sampling probe 11B are interchangeable on the same device 10.

In the illustrated embodiment in FIGS. 1 to 3 and 6 to 8, the soil probe 11 is a soil sensor probe 11A. A lower portion 17 of the soil sensor probe 11A is equipped with a plurality of sensors for sensing soil properties. The sensors include a soil electrical conductivity (EC) sensor 18 for determining soil texture, a soil optical reflectance sensor 19 for determining soil organic carbon or soil organic matter, and a soil capacitance sensor 20 for determining soil moisture. The soil probe 11 also includes a soil probe insertion force sensor 21 for determining soil compactness. The soil optical reflectance sensor 19 preferably senses soil reflectance at two wavelengths to more accurately measure soil organic carbon/matter. The soil sensors 18, 19 and 20 are down hole, in situ sensors on the sensor probe 11A that collect soil data at multiple depths as the sensor probe 11A is being inserted into the soil to develop a profile of the soil properties.

Sensor combinations can estimate soil bulk density using a pedotransfer function. Soil bulk density is strongly affected by soil texture, soil organic carbon/matter, soil moisture, and soil compactness. Pedotransfer functions can use various regression analysis and data mining techniques to associate these soil properties at each sample zone with an estimated soil bulk density at that location.

In the illustrated embodiment in FIGS. 9 and 10, the soil probe 11 is a soil core sampling probe 11B for collecting soil core samples at selected sampling locations. The soil core sampling probe 11B has a cutting shoe 61 at a lower end, a core sampler tube 62, and a core sampler tube liner 63. A quick attach coupling 64 is provided at an upper end of the soil core sampling probe 11B for attaching to a ratcheting rotational mounting head 65. A ratchet handle 66 is provided on the mounting head 65 for rotating the quick attach coupling 64 and the core sampler tube 62. The core samples collected by the soil core sampling probe 11B can be used for laboratory analysis of soil properties (e.g., soil pH, nutrient levels, etc.) and for calibrating the sensors on the soil sensor probe 11A. The quick attach coupling 64 and mounting head 65 illustrated in FIG. 10 can be used to interchangeably mount the soil sensing probe 11A and the soil core sampling probe 11B to the shuttle system 14.

The linear actuator 12 is a first hydraulic actuator connected to a source of hydraulic pressure, such as a remote hydraulic port on the vehicle. For example, the linear actuator 12 can be a 30 inch hydraulic cylinder. A solenoid controlled hydraulic control system 22 is provided to control fluid flow to the hydraulic cylinder 12 through hydraulic lines 23. The movement of the hydraulic cylinder 12 and the amount of downforce applied can be manually or automatically controlled by the hydraulic control system 22.

The insertion force sensor 21 provides a first force measurement sensor associated with the hydraulic actuator 12 for measuring a downforce applied to the soil probe 11. For example, the first force measurement sensor 21 can be a strain gauge in the probe housing to measure insertion force directly. A second force measurement sensor 24 associated with the hydraulic actuator 12 is a transducer in the hydraulic line 23 or in the hydraulic control system 22 that measures the hydraulic pressure being applied to the hydraulic actuator 12. The hydraulic pressure in the hydraulic actuator 12 provides an indirect measurement of the downforce being applied to the soil probe 11. A combination of the strain gauge sensor 21 in the probe housing and the transducer 24 in the hydraulic line can be used to measure the downforce applied to the soil probe 11.

The hydraulic control system 22 has at least one pulse width modulated (PWM) control valve for applying controlled hydraulic force to the linear actuator 12 from the source of hydraulic pressure. The PWM control valve allows operator selectable, accurate and efficient soil probe insertion speeds and rapid soil probe retraction speeds with various hydraulic outputs.

The soil engaging foot 13 is attached to a mast frame 25 of the device 10 with a pair of vertical guide rails 26, 27 that allow the soil engaging foot 13 to move vertically relative to the mast frame 25. Coil springs 28, 29 are arranged on each of the vertical guide rails 26, 27 to bias the soil engaging foot 13 downward to its lowest position relative to the mast frame 25. The soil engaging foot 13 has a centering hole 30 for guiding the soil probe 11 into the soil.

A second hydraulic actuator 31 is provided to move the mast frame 25 vertically relative to a base frame 32 connected to the mounting hitch 16. The mast frame 25 can be moved vertically by the second hydraulic actuator 31 between a raised transport position and a lowered working position. When the mast frame 25 is moved to its lowered working position, the soil engaging foot 13 is pressed against the soil surface S causing the coil springs 29 and 30 to be compressed as the soil engaging foot 13 and mast frame 25 move relative to each other. A second force measurement sensor 33 measures the hydraulic pressure in the hydraulic line to the second hydraulic actuator 31 to measure the downforce applied to the soil engaging foot 13.

A depth gauge plate 34 is secured to one of the vertical guide rails 26 and extends upwardly along a vertical length of the mast frame 25. Since the depth gauge plate 34 is fixed relative to the soil engaging foot 13 via the guide rail 27, the depth gauge plate 34 can be used to provide an accurate position measurement relative to the soil surface S when the soil engaging foot 13 is in contact with the soil surface S.

A position sensor 35 is provided at an upper end of the depth gauge plate 34 to detect the relative position of the soil engaging foot 13 relative to the mast frame 25. For example, the position sensor 35 can be a rotary sensor connected by a linkage 36 to the depth gauge plate 34 that outputs a signal when the depth gauge plate 34, and hence the soil engaging foot 13, moves vertically relative to the mast frame 25 at least a predetermined distance. When the mast frame 25 is lowered to its working position, and the soil engaging foot 13 is pressed against the soil surface S, the coil springs 28, 29 are compressed, the depth gauge plate 34 moves relative to the mast frame 25, and the position sensor 35 will provide a signal indicating that the soil engaging foot 13 is being pressed against the soil surface. The hydraulic control 37 for the second hydraulic actuator 31 will then stop further downward movement, hold the mast frame 25 in its lowered working position, and allow the probe operating sequence to continue by inserting the soil probe 11 through the centering hole 30 in the soil engaging foot 13 and into the soil.

The vibration mechanism 15 is arranged to apply vibration force through the soil probe 11 together with the downforce applied by the linear actuator 12 to facilitate insertion of the soil probe 11 into the soil. The vibration mechanism 15 ensures adequate penetration of the soil probe 11 while maintaining a lightweight and compact configuration.

The shuttle system 14 has a multiple hit configuration that allows insertion of the soil probe 11 into the soil to a depth that exceeds a stroke length of the linear actuator 12. The shuttle system 14 allows the device 10 to have a more compact design by shortening the required stroke length of the linear actuator 12 to accomplish the desired probe insertion depth. For example, a 40 inch probe insertion depth can be accomplished with a 30 inch cylinder using a double hit shuttle configuration.

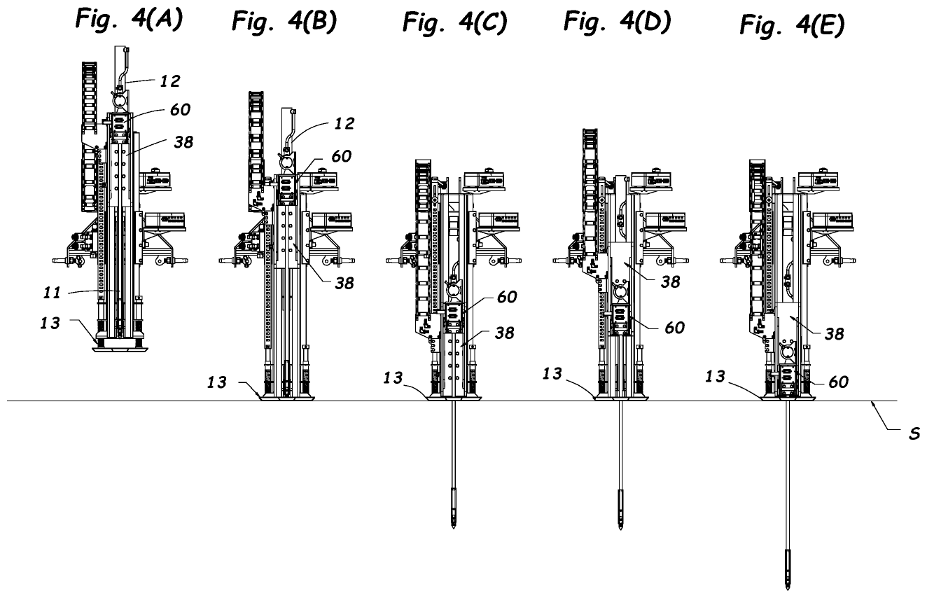

The operation of the shuttle system 14 is illustrated in FIGS. 4(A) to 4(E). The double hit shuttle configuration is accomplished by having the probe carrier 38 coupled with an upper end 60 of the soil probe 11 using mechanically or electrically actuated locking pins 50, 51 that selectively lock the probe carrier 38 relative to the soil probe 11 in either a shallow stage insertion/retraction position or a deep stage insertion/retraction position. The locking pins 50, 51 can be automatically operated by the controller 39.

FIG. 4(A) shows the soil probe 11, foot 13 and probe carrier 38 in a raised transport position. FIG. 4(B) shows the soil probe 11, foot 13 and probe carrier 38 lowered with the foot 13 in a compressed position against the soil surface S and the soil probe 11 and probe carrier 38 in a top carrier position. FIG. 4(C) shows the soil probe 11 in a first probe stage position in which the probe carrier 38 has been moved to a lowered position and the soil probe 11 is inserted into the soil to a first depth. The soil probe 11 is then unlocked from the probe carrier 38, and the probe carrier 38 is raised relative to the soil probe 11 to engage the soil probe in a second locked position, as illustrated in FIG. 4(D). The probe carrier 38 and soil probe 11 are then lowered to a final probe stage with the soil probe 11 fully inserted into the soil, as illustrated in FIG. 4(E).

Once the lower extent of the final probe stage is reached, the probe 11 and probe carrier 38 are raised, and the process is reversed to extract the soil probe 11 from the soil. The multiple hit configuration of the shuttle system 14 provided by the multiple stages of the probe carrier 38 allows a more compact configuration of the device 10 without having the probe 11 depth limited to the stroke length of the linear actuator 12.

The soil profile sensing device 10 includes a controller 39 with a microprocessor programmed to perform a pedotransfer function to estimate soil bulk density based on soil data collected by the sensors 18-21 on the soil probe 11.

The controller 39 receives position signals from an optical sensor assembly 40 that detects vertical movement and vertical positions of the soil probe 11 and the shuttle system 14. The optical sensor assembly 40 provides directional (i.e., up/down) information needed for automation of the probe sampling sequence.

The optical sensor assembly 40 has first and second optical sensors 41, 42 mounted to move up and down with the soil probe 11 relative to the depth gauge plate 34. The depth gauge plate 34 has a plurality of vertically spaced openings 43 along a length thereof to form a depth ruler. The optical sensors 41, 42 each include a transmitter and a receiver positioned on opposite sides of the depth ruler 34, with one optical sensor 41 being positioned slightly higher than the other optical sensor 42. The vertically spaced openings 43 in the depth ruler 34 allow light emitted by the transmitters of the optical sensors 41, 42 to be received by the receivers of the optical sensors 41, 42, and a corresponding signal to be sent to the controller 39. The optical sensors 41, 42 detect a direction and distance of movement of the optical sensors 41, 42 along the depth ruler 34, which directly corresponds to a depth of insertion of the soil probe 11 into the soil when the foot 13 is pressed against the soil surface S.

The optical sensor assembly 40 also has a third optical sensor 44 mounted to move up and down with the soil probe 11 relative to the depth gauge plate 34. The depth gauge plate 34 has an upper limit opening 45 that aligns with the third optical sensor 44 when the soil probe 11 is in its fully raised position. The third optical sensor 44 includes a transmitter and a receiver positioned on opposite sides of the depth ruler 34, and the upper limit opening 45 allows light emitted by the transmitter to be received by the receiver of the third optical sensor 44, and a corresponding signal to be sent to the controller 39 indicating the soil probe 11 is in its fully raised position.

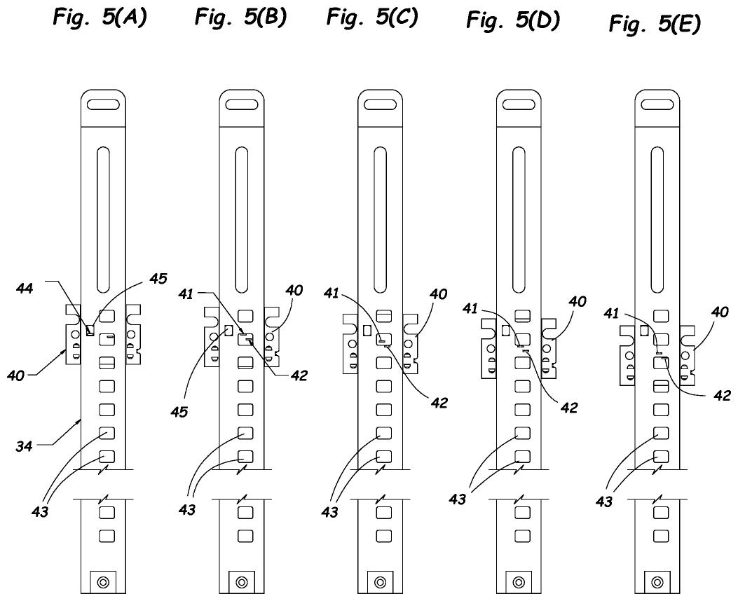

In FIG. 5(A), the third optical sensor 44 is shown in a full up position of the soil probe 11 with the upper limit or “probe up” opening 45 in the depth ruler 34 allowing the third optical sensor 44 to detect that the shuttle system 14 is in the full up position and send a position signal to the controller 39. Also shown in FIG. 5(A) is a bottom receiver of the second optical sensor 42 aligned with one of the vertically spaced openings 43 in the depth ruler 34.

In FIG. 5(B), the first and second optical sensors 41, 42 are shown in a slightly lowered position relative to the depth ruler 34. In this position, top and bottom receivers of the optical sensors 41, 42 are aligned with the opening 43 in the depth ruler 34.

In FIG. 5(C), the first and second optical sensors 41, 42 are shown in a slightly lowered position relative to the depth ruler 34, as compared to FIG. 5(B). In this position, the top receiver of the optical sensor 41 is aligned with the opening 43 in the depth ruler 34, but the bottom receiver of the optical sensor 41 is not aligned with the opening 43.

In FIG. 5(D), the first and second optical sensors 41, 42 are shown in a slightly lowered position relative to the depth ruler 34, as compared to FIG. 5(C). In this position, neither of the top and bottom receivers of the optical sensors 41, 42 are aligned with the opening 43 in the depth ruler 34.

In FIG. 5(E), the first and second optical sensors 41, 42 are shown in a slightly lowered position relative to the depth ruler 34, as compared to FIG. 5(D). In this position, the top receiver of the optical sensor 41 is not aligned with the opening 43 in the depth ruler 34, but the bottom receiver of the optical sensor 41 is aligned with the opening 43.

As explained above, FIGS. 5B, 5(C), 5(D) and 5(E) illustrate positions of the top and bottom receivers as the optical sensors 41, 42 continue to move lower along the depth ruler 34. As the top and bottom receivers of the optical sensors 41, 42 move into and out of alignment with the vertically spaced openings 43 in the depth ruler 34, corresponding signals are sent to the controller 39 to monitor the direction and distance of movement of the optical sensors 41, 42 along the depth ruler.

A GPS or other suitable position detecting system is provided in the controller 39 to determine a position of the soil probe device 10 within a field and a horizontal speed of the soil probe device 10. A sampling distance interval or sampling grid size can be input by a user to set the distance between sampling locations. The detected position of the soil probe device 10 in the field will be processed with the sampling distance interval to determine where the soil probe device 10 should be moved for each sampling location.

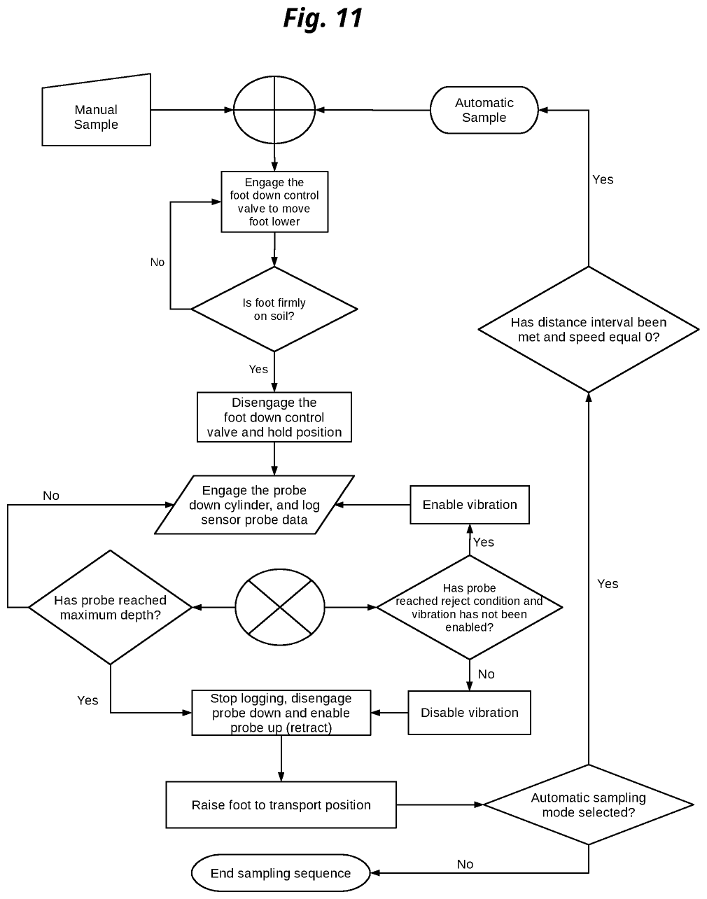

FIG. 11 is a flowchart of a probe sampling sequence programmed into the controller 39. The probe sampling sequence can be used to automatically insert the soil probe 11 into the soil to sense one or more soil properties or collect a soil core without supervision or manual control by the operator. The automatic probe sampling sequence is initiated when the sampling distance interval has been met and the horizontal speed of the soil probe device 10 is determined to be approximately zero (i.e., the horizontal movement has stopped). The automatic probe sampling sequence allows for rapid and repetitive insertions of the soil probe 11 into the soil at several sampling locations across the field for complete characterization of the soil profile in the field.

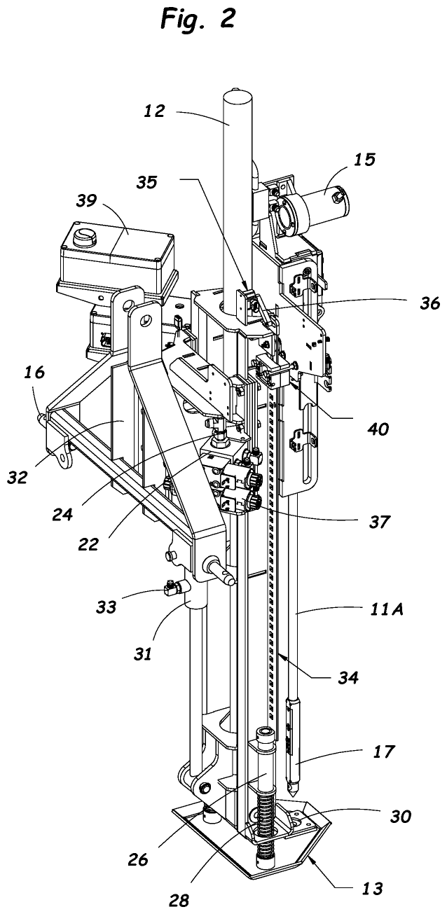

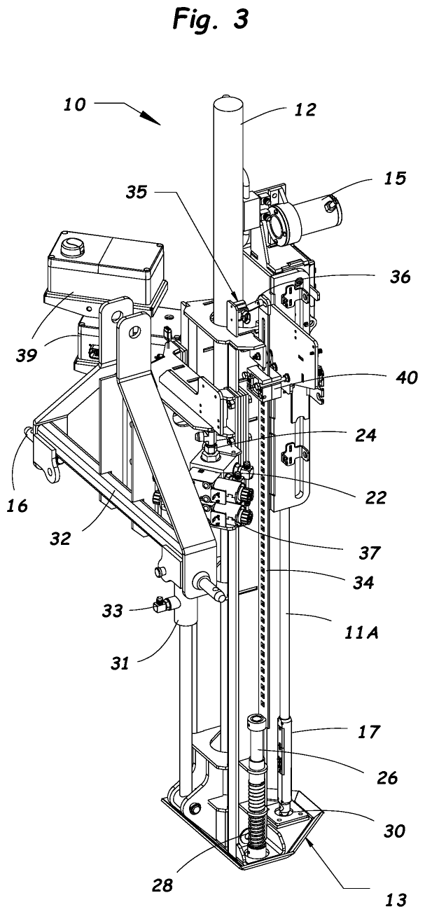

Upon initiating the automatic probe sampling sequence, the soil engaging foot 13 is lowered into engagement with the soil surface. The rotary foot position sensor 35 determines whether the foot 13 is in an uncompressed condition (FIG. 2) or a compressed condition (FIG. 3). The hydraulic pressure sensor 33 provides a measurement of how much downforce is applied to the foot 13. The foot position sensor 35 and hydraulic pressure sensor 33 together determine when the foot 13 is firmly engaged with the soil surface.

Once the foot 13 is determined to be firmly engaged with the soil surface S, the hydraulic cylinder 12 of the downforce mechanism is actuated to apply a downforce to the soil probe 11. The soil probe 11 is inserted through the centering hole 30 of the foot 13 into the soil. As the soil probe 11 is being inserted into the soil, the depth of the soil probe 11 is measured by the optical sensors 41, 42 moving along the depth ruler 34.

If soil conditions are encountered that require excessive downforce to penetrate with the soil probe 11, the vibration mechanism 15 is manually or automatically activated to facilitate insertion of the probe 11 into the soil. The vibration mechanism 15 can be automatically activated based on one or more of the following conditions: (1) a detected pressure in the hydraulic cylinder 12 exceeding a predetermined pressure; (2) a force detected by the load transducer or strain gauge 21 associated with the soil probe 11 exceeding a predetermined force; and (3) the position sensor 35 attached to the foot 13 indicating the foot 13 is no longer in contact with the soil.

The automatic probe sampling sequence also controls the shuttle system 14 to transfer downforce from the linear actuator 12 to the soil probe 11. The shuttle system 14 engages the soil probe 11 in the first stage of insertion to a depth corresponding to the stroke length of the linear actuator 12. The shuttle system 14 then disengages from the soil probe 11 and is raised to a position where it re-engages the soil probe 11 and starts a second stage of insertion. The second stage of insertion pushes the soil probe 11 to a depth that exceeds the stroke length of the linear actuator 12.

After the soil probe 11 has been fully inserted into the soil to a preset or maximum sensing or sampling depth, the controller 39 actuates the linear actuator 12 to retract the soil probe 11 back to a raised transport position. The shuttle system 14 is operated in reverse order to cause the soil probe 11 to be raised in multiple stages back to its raised transport position.

The pressure sensors 24, 33 and position sensors 41, 42, 44 used in the soil probing device 10 eliminate the need for operator supervision at each stage of the sampling process. The automatic mode can be set to automatically insert the soil probe 11 into the soil as soon as the vehicle stops, and also to automatically engage the vibration mechanism 15 when needed for the probe 11 to penetrate into hard-to-penetrate soil. An auto reject feature will cause the controller 39 to stop the probe insertion upon encountering conditions in which a maximum downforce or hydraulic pressure threshold is met or exceeded.

Data collection software with an X-Y sensor map and sample point guiding can be used to facilitate automated collection of soil data with the soil probing device 11. A data log display can be used to provide real time information about the soil profile during the probe sampling sequence.

Post-processing routines can also be performed on the collected data, including themed probe insertion graphs. For example, the associated software can provide hover over or click display graphs to provide soil profile information from the resulting soil maps.

The soil probing device 10 of the present invention provides advantages over existing soil sensing and sampling devices. For example, the soil probing device 10 is more compact than existing soil sensing and sampling devices, which typically need a tractor or large 4WD pickup to support them. Moreover, even though the soil probing device 10 is small, the vibration mechanism 15 gives the soil probing device 10 a sufficient penetration depth and capability to penetrate hard soil.

The automation provided by the soil probing device 10 allows rapid, complete profile characterization of a field without the operator leaving the seat. The soil probing device 10 could also be robotic controlled.

The moisture sensor 20 used in the soil sensor probe 11A in conjunction with other sensors can be calibrated to soil bulk density. The soil bulk density calculation allows the present invention to provide improved soil organic carbon measurements for conservation practices and SOC marketing.

Future improvements to the soil probing device 10 are contemplated. For example, additional sensors can be used with the soil probing device 10, including cameras and microscopes. Stratification software can be used with X-Y type maps and algorithms to select optimal core probing locations for sensor calibrations.

Sensors can also be provided on the soil core sampler 11B, similar to the soil sensor probe 11A. Such sensors would improve the sample results and aid in SOC and soil bulk density calibrations.

Additional software related features can be incorporated into the automated controller 39. FIG. 10 is a flowchart for an “erroneous log detector” that can be provided to flag the operator when the probe data is demonstrably different than the other insertions in the field. For example, the controller 39 can be programmed to flag the operator if the one of the detected soil properties is higher or lower than a field average by more than a predetermined percentage. Upon receiving a signal from the erroneous log detector, the operator will be prompted to take another insertion nearby in the same sampling zone to replace the soil data determined to be erroneous. The automated controller 39 can also be programmed to automatically insert the soil probe 11 nearby in the same sampling zone to collect data to replace the erroneous soil data.

The controller 39 can be programmed to provide sampling site suggestions for taking soil core samples for calibration purposes. The controller software examines the logs from the field and guides the operator to sites for core sampling that will provide the most significant variability in the soil properties. Such variability is important for high quality calibrations.

The controller 39 can have a setup screen and function to allow the user to select a maximum probing depth to ensure safe operations. For example, since the operator could encounter gas, electrical, or communication lines in probing some areas, the system will require the operator to enter a maximum depth for the insertions and confirm that the operator has verified there are no underground issues for the site. The system will then only probe to the maximum depth set by the user, thereby ensuring safety and minimizing damage to underground utilities and the like.

The setup screen and function of the controller 39 can be used to set the maximum downpressure for the probe device 10. For example, the probe device 10 may be built to handle 12,000 kilopascals (kpa) when mounted to a compact tractor, but when the probe device is attached to a utility vehicle the maximum downpressure on the probe device may only be about 7500 kpa due to the lighter weight of the vehicle.

The data collection software in the controller 39 can also import other soil data to aid in the data collection. For example, if a geo-referenced topographic map or other geo-referenced map of previously collected soil properties has already been generated for a field, that data can be imported and used to help determine the optimum sampling locations or to resolve other soil properties based on the soil data currently being collected.

The data collection software in the controller 39 can also be used to measure the topsoil depth, for example, by mining the first derivatives of the various sensors. Topsoil can typically be distinguished from subsoil by visual characteristics that correlate to differences in soil reflectance. For example, a topsoil layer can typically be distinguished from the underlying subsoil by color changes in the physical soil core and a corresponding increase in soil reflectance.

The term “utility vehicle” in this application refers to a utility terrain vehicle (UTV) or a multipurpose off-highway utility vehicle (MOHUV), as defined by the American National Standards Institute (ANSI), and similar vehicles. The term “compact tractor” in this application refers to a small agricultural tractor designed for Category I implements or a compact utility tractor, as defined by the American Society of Agricultural Engineers (ASAE), and similar vehicles.

While the invention has been described in connection with specific embodiments thereof, it is to be understood that this is by way of illustration and not of limitation, and the scope of the appended claims should be construed as broadly as the prior art will permit.

What is claimed is:

1. A soil profile sensing device, comprising:
a soil probe equipped with a plurality of sensors for sensing a plurality of soil properties selected from the group consisting of soil EC, soil optical reflectance, soil capacitance, and soil compactness; and
an automated controller that automatically inserts the soil probe into the soil upon detecting that a predetermined sampling distance interval has been met and the soil probe has stopped horizontal movement.

2. The soil profile sensing device according to claim 1, wherein said plurality of sensors comprises a soil EC sensor for determining soil texture.

3. The soil profile sensing device according to claim 1, wherein said plurality of sensors comprises a soil optical reflectance sensor for sensing soil reflectance at two wavelengths to determine soil organic carbon or soil organic matter.

4. The soil profile sensing device according to claim 1, wherein said plurality of sensors comprises a soil capacitance sensor for determining soil moisture.

5. The soil profile sensing device according to claim 1, wherein said plurality of sensors comprises a soil probe insertion force sensor for determining soil compactness.

6. The soil profile sensing device according to claim 5, wherein said plurality of sensors comprises a soil EC sensor for determining soil texture, a soil optical reflectance sensor for sensing soil reflectance at two wavelengths to determine soil organic carbon or soil organic matter, a soil capacitance sensor for determining soil moisture, and a soil probe insertion force sensor for determining soil compactness.

7. The soil profile sensing device according to claim 6, further comprising a means for determining soil bulk density using soil data collected by said plurality of sensors.

8. The compact soil probing device according to claim 7, wherein said means for determining soil bulk density comprises a processor programmed to perform a pedotransfer function to estimate soil bulk density based on soil data collected by said plurality of sensors.

9. The compact soil probing device according to claim 1, further comprising a spring-loaded, soil engaging foot with a centering hole for guiding said soil probe into the soil.

10. The compact soil probing device according to claim 9, wherein said automated controller detects whether the spring-loaded foot is firmly on the soil surface before inserting the soil probe into the soil.

11. The compact soil probing device according to claim 1, wherein said automated controller activates a vibration mechanism when needed to facilitate insertion of the soil probe into the soil.

12. A compact soil probing device, comprising:
a soil probe;
a linear actuator for applying downforce to push the soil probe into soil; and
a shuttle system arranged to transfer downforce from said linear actuator to said soil probe, said shuttle system having a multiple hit configuration that allows insertion of the soil probe into the soil to a depth that exceeds a stroke length of said linear actuator.

13. The compact soil probing device according to claim 12, wherein said soil probe comprises a soil sensor probe equipped with at least one sensor for sensing a soil property selected from the group consisting of soil EC, soil optical reflectance, soil capacitance, and soil compactness.

14. The compact soil probing device according to claim 12, wherein said soil probe comprises a soil core sampling probe for collecting soil core samples.

15. The compact soil probing device according to claim 12, wherein said soil probing device is adapted to be mounted to a utility vehicle or a compact tractor.

16. The compact soil probing device according to claim 12, further comprising a spring-loaded, soil engaging foot with a centering hole for guiding said soil probe into the soil.

17. The compact soil probing device according to claim 12, wherein said linear actuator is a hydraulic actuator connected to a source of hydraulic pressure.

18. The compact soil probing device according to claim 17, further comprising a first force measurement sensor associated with said hydraulic actuator for measuring a downforce applied to said soil probe.

19. The compact soil probing device according to claim 18, further comprising a second force measurement sensor associated with said spring-loaded foot for measuring a downforce on said spring-loaded foot.

20. The compact soil probing device according to claim 17, further comprising a vibration mechanism for applying vibration force together with the downforce applied by said linear actuator to facilitate insertion of the soil probe into the soil.

21. The compact soil probing device according to claim 17, further comprising at least one PWM controlled valve for applying controlled hydraulic force to said linear actuator from said source of hydraulic pressure, said PWM controlled valve allowing operator selectable, accurate and efficient soil probe insertion speeds and rapid soil probe retraction speeds.

22. The compact soil probing device according to claim 12, further comprising a plurality of optical sensors on a depth gauge plate for detecting vertical positions of said soil probe or said shuttle system.

23. A method of operating a soil probe device, comprising:
determining a location of the soil probe device within a field using a GPS receiver;
selecting a sampling distance interval;
measuring a horizontal speed of the soil probe device;
initiating an automatic probe sampling sequence when the sampling distance interval has been met and the horizontal speed of the soil probe device is zero; and
operating the soil probe device according to the automatic probe sampling sequence to insert a soil probe of the soil probe device into soil to sense a soil property or collect a soil core.

24. The method of operating a soil probe device according to claim 23, wherein said soil probe device comprises a soil engaging foot with a centering hole for guiding the soil probe into the soil, and wherein said automatic probe sampling sequence comprises lowering the soil engaging foot into engagement with the soil surface, and actuating a downforce mechanism to apply a downforce to the soil probe to insert the soil probe through the centering hole into the soil.

25. The method of operating a soil probe device according to claim 24, wherein said automatic probe sampling sequence further comprises determining when the soil engaging foot is firmly engaged with the soil surface by sensing a position of the soil engaging foot and a pressure of a hydraulic actuator that lowers the soil engaging foot into engagement with the soil surface.

26. The method of operating a soil probe device according to claim 23, wherein said automatic probe sampling sequence further comprises retracting the soil probe to a transport position after the soil probe has been inserted into the soil to a preset or maximum depth.

27. The method of operating a soil probe device according to claim 23, wherein said automatic probe sampling sequence further comprises activating a vibration mechanism to apply vibration to the soil probe to facilitate insertion of the soil probe into the soil.

28. The method of operating a soil probe device according to claim 27, wherein said step of activating a vibration mechanism comprises automatically activating the vibration mechanism based on one or more of the following conditions: a detected pressure in the downforce mechanism exceeding a predetermined pressure, a force detected by a transducer or strain gauge associated with the soil probe exceeding a predetermined force, and a position sensor attached to the foot indicating the foot is no longer in contact with the soil.

29. The method of operating a soil probe device according to claim 23, wherein the soil probe device is used for sensing at least one soil property selected from the group consisting of soil EC, soil optical reflectance, soil capacitance, and soil compactness.

30. The method of operating a soil probe device according to claim 23, wherein the soil probe device is used for collecting soil core samples.

31. The method of operating a soil probe device according to claim 23, wherein said automatic probe sampling sequence further comprises using a shuttle system to transfer downforce from a linear actuator to the soil probe, and operating the shuttle system to insert the soil probe into the soil to a depth that exceeds a stroke length of the linear actuator.

32. The method of operating a soil probe device according to claim 23, further comprising using at least one PWM controlled valve to apply controlled hydraulic force 2 to the linear actuator from a source of hydraulic pressure to provide operator selectable soil probe insertion and retraction speeds.

33. The method of operating a soil probe device according to claim 23, wherein said soil probing device is mounted to a utility vehicle or a compact tractor.
